(19) United States
(12) Patent Application Publication
(10) Pub. No.: US 2018/0168182 A1
(43) Pub. Date: Jun. 21, 2018

(54) CHEWABLE COMPOSITIONS CONTAINING CURCUMINOIDS AND THEIR METHOD OF PREPARATION

(71) Applicants: Muhammed Majeed, Edison, NJ (US); Kalyanam Nagabhushanam, East Windsor, NJ (US); Anurag Pande, East Windsor, NJ (US); Shaheen Majeed, Springville, UT (US)

(72) Inventors: Muhammed Majeed, Edison, NJ (US); Kalyanam Nagabhushanam, East Windsor, NJ (US); Anurag Pande, East Windsor, NJ (US); Shaheen Majeed, Springville, UT (US)

(21) Appl. No.: 15/846,385

(22) Filed: Dec. 19, 2017

Related U.S. Application Data
(60) Provisional application No. 62/437,652, filed on Dec. 21, 2016.

(51) Int. Cl. A23G 3/48; A23G 3/42; A23P 30/10; A23P 20/10; A61K 36/9066

(52) U.S. Cl. CPC A23G 3/48; A23V 2300/36; A23P 30/10; A23P 20/10; A61K 36/9066; A23G 2200/06; A23V 2002/00; A23V 2200/06; A23V 2200/15; A23V 2250/2112; A23V 2250/032; A23V 2250/502; A23V 2250/606; A23V 2250/61; A23V 2250/616; A23V 2250/628; A23V 2300/31; A23G 3/42

(57) ABSTRACT

Disclosed is a method for the preparation of a gummy and candy composition containing curcuminoids. Also disclosed is a gummy and candy composition containing effective concentration of curcuminoids that is palatable and bioavailable.

### BACKGROUND OF THE INVENTION

### Field of the Invention

The present invention in general relates to confectioneries. More specifically, the present invention relates to chewable compositions containing curcuminoids. The invention also relates to a process of preparing chewable compositions containing curcuminoids.

### Description of Prior Art

Curcuminoids, from Curcuma longa, are being widely used as a dietary supplement owing to their increased health benefits. They are being effectively administered in the form of tablets, pills, powder, capsules etc. Due to their peculiar flavour, a more palatable form of administration is required to increase its reach as a dietary supplement to a wide range of consumers. EP2559347 discloses a composition that masks the flavour of curcumin. The patent also suggests room for improvement in their flavour masking effects. Another important aspect to be addressed is the bioavailability and solubility of curcuminoids. US 2016/0374962 discloses a microsized curcumin composition with higher bioavailability to treat diseases such as head and neck disorders and upper aerodigestive disorders. But there still exist an unmet industrial need to produce a more palatable and bioavailable curcuminod composition. The present invention solves the above technical problem by disclosing a curcuminoid gummy and candy composition that is more palatable and bioavailable. The invention also discloses a novel process to produce the abovementioned composition.

The principle objective of the inventions is to disclose a gummy and candy composition comprising curcminoids that is more palatable and bioavailable.

It is yet another objective of the invention to disclose a novel process to manufacture the curcuminoid composition that is more palatable and bioavailable.

The present invention fulfils aforesaid objectives and provides further related advantages.

### SUMMARY OF THE INVENTION

The present invention discloses a novel process for the manufacture of a gummy and candy composition containing curcuminoids. The invention also discloses a gummy and candy composition containing curcuminoids that is palatable and bioavailable.

### DESCRIPTION OF THE MOST PREFERRED EMBODIMENTS

In a principle embodiment, the present invention discloses a method for the manufacture of gummy composition containing curcuminoids, said method comprising steps of:
a) Mixing a powdered chewable base in water with constant stirring to obtain a turbid solution;
b) Adding appropriate concentration of sweetners to the solution of step a with continuous stirring;
c) Mixing the solution of step b with suitable acidifying agents and flavouring agents to obtain a colloidal mixture;
d) Heating the mixture of step c to 55° C.-80° C. for 15-20 minutes
e) Adding an effective concentration of curcumnioids to the solution of Step d with continuous stirring to form a uniform mixing of the ingredients
f) Pouring the syrup of step e into moulds of different shapes and sizes that is precoated with corn starch
g) Allowing the syrup to cool at 10-15° C. within the mould and solidify to obtain a chewable gummy
h) Passing the gummies of step g on Vibrosifters to remove excess corn starch
i) Sprinkling suitable sweetners, acidifying agents and flavouring agents as a coating.
j) Packing the solidified chewable candy into suitable containers and packets

In a preferred embodiment, the chewable base is selected from the group consisting of gelatin, soft gum, nougat and caramel. In a related embodiment the chewable base is present at concentrations of 5-80% w/w. In another preferred embodiment, the curcuminoids are present at concentrations of 10-250 mg and 1-20% w/w of the total composition. In another preferred embodiment, the ratio of curcuminoids is 75-81% curcumin, 15-19% demethoxy curcumin, and 2.2-6.5% bis demethoxy curcumin. In another related embodiment, the sweetner is selected from the group consisting of aspartame, sucrose, fructose, glucose, corn syrup, maltose, dextrose, monk fruit extract and sugar substitutes like stevia at concentrations of 30-70% w/w of the total composition. In another related embodiment, the acidifying agent is selected from the group consisting of citric acid, tartaric acid, fumaric acid and lactic acid, at concentrations of 0.5-10% w/w of the total composition. In another related embodiment, the flavouring agent is selected from the group consisting of natural flavours obtained from fruits, berries, honey, molasses, maple sugar, or artificial flavours: methyl anthranilate and ethyl caproate, at concentrations of 0.5-10% w/w of the total composition.

In another related embodiment, the present invention also discloses a chewable gummy composition comprising of effective concentration of curcuminoids, chewable base, suitable lubricants, acidifying agents, preservatives and flavouring agents. In a related preferred embodiment, the aforementioned composition is more palatable and bioavailable. In another preferred embodiment, the ratio of curcuminoids is 75-81% curcumin, 15-19% demethoxy curcumin, and 2.2-6.5% bis demethoxy curcumin.

In another preferred embodiment, the present invention discloses a method for the manufacture of a candy composition containing curcuminoids, said method comprising steps of:
a) Mixing one or more sweetners in water in a syrup preparation tank
b) Heating the mixture to 100-110° C. to prepare a clear syrup
c) Transferring the syrup of step b to main cooking vessel and heating to 130-150° C.
d) Vaccumising the bulk (800-900 mbar) to achieve water content below 2.5%.
e) Adding an effective concentration of curcumnioids, acidifying and flavouring agents to step d and heating to 125-135° C.
f) Mixing the solution of step e to form a uniform homogenous mixture
g) Subjecting the mixture of step f to cooling & kneading
h) Forwarding the mixture of step g to batch former followed by rope former
i) Passing the rope through candy formation machine to mould the candies
j) Passing the moulded candies through a cooling tunnel at 2-8° C.
k) Packing the solidified chewable candy into suitable containers and packets

In another preferred embodiment, the curcuminoids are present at concentrations of 10-100 mg and 1-20% w/w of the total composition. In another preferred embodiment, the ratio of curcuminoids is 75-81% curcumin, 15-19% demethoxy curcumin, and 2.2-6.5% bis demethoxy curcumin. In another related embodiment, the sweetner is selected from the group consisting of aspartame, sucrose, fructose, glucose, corn syrup, maltose, dextrose, monk fruit extract and sugar substitutes like stevia at concentrations of 30-80% w/w of the total composition. In another related embodiment, the acidifying agent is selected from the group consisting of citric acid, tartaric acid, fumaric acid and lactic acid, at concentrations of 0.1-10% w/w of the total composition. In another related embodiment, the flavouring agent is selected from the group consisting of natural flavours obtained from fruits, berries, honey, molasses, maple sugar, or artificial flavours: methyl anthranilate and ethyl caproate, at concentrations of 0.1-10% w/w of the total composition.

In another related embodiment, the present invention also discloses a chewable candy composition comprising of effective concentration of curcuminoids, chewable base, suitable lubricants, acidifying agents, preservatives and flavouring agents. In a related preferred embodiment, the aforementioned composition is more palatable and bioavailable. In another preferred embodiment, the ratio of curcuminoids is 75-81% curcumin, 15-19% demethoxy curcumin, and 2.2-6.5% bis demethoxy curcumin.

The aforesaid most preferred embodiments incorporating the technical features and technical effects of instant invention, are explained through illustrative examples herein under.

### Example 1: Method of Preparing a Curcuminoid Gummy Composition

The present invention provides for suitably flavored chewable soft gummy made by using curcuminoids enriched turmeric extract to deliver flavored brand confection product containing minimum 250 mg of bioavailable Curcuminoids per gummy obtained from turmeric roots. The present invention can have gelatin or pectin as a base, along with emulsifier, pH modifier, natural flavor like mango, refined sugar and corn syrup and preservative. The present invention was created to overcome the organoleptic, poor water solubility and bioavailability challenges in curcuminoids supplementation. The present invention provides therapeutically active dosage of curcuminoids obtained from turmeric extract. To enhance the acceptance to the high dosage of the curcuminoids in the gummy, the said gummies were sprinkled with tartatic acid and refined sugar. The present invention also provides stable composition of curcuminoids containing minimum 75-81% curcumin, 15-19% demethoxy curcumin, and 2.2-6.5% bis demethoxy curcumin in each gummy. The invention deals with flavored gummy confection with improved bioavailability and taste profile and hence health characteristics of curcuminoids based turmeric extract.

The method of preparing the gummy includes the following steps:
a) Mixing a gelatin base in water with constant stirring to obtain a turbid solution;
b) Adding appropriate concentration of glucose corn syrup and refinated sugar to the solution of step a with continuous stirring;
c) Mixing the solution of step b with suitable citric acid, lactic acid and natural mango flavour M38630 to obtain a colloidal mixture;
d) Heating the mixture of step c to 55° C.-80° C. for 15-20 minutes
e) Adding an effective concentration of curcumnioids to the solution of Step d with continuous stirring to form a uniform mixing of the ingredients
f) Pouring the syrup of step e into moulds of different shapes and sizes that is precoated with corn starch
g) Allowing the syrup to cool at 10-15° C. within the mould and solidify to obtain a chewable gummy
h) Passing the gummies of step g on Vibrosifters to remove excess corn starch
i) Sprinkling suitable tartaric acid, and refinated sugar as a coating
j) Packing the solidified chewable candy into suitable containers and packets
   Table 1 provides an illustrative examples of a curcuminoid gummy formulation

| | | |
|---|---|---|
| 1 | GELATIN (270 BLOOM MESH 10) | 6.00% |
| 2 | REFINATED SUGAR | 28.24% |
| 3 | GLUCOSE CORN SYRUP | 37.29% |
| 4 | CITRIC ACID | 0.90% |
| 5 | LACTIC ACID | 0.80% |
| 6 | WATER | 8.66% |
| 7 | CURCUMIN C3 COMPLEX ® | 7.81% |
| 8 | NATURAL MANGO FLAVOR M38630 | 0.30% |
| 9 | DL-TARTARIC ACID | 0.20% |
| 10 | REFINATED SUGAR | 9.80% |
| | TOTAL | 100.00% |

### Example 2: Method of Preparing a Curcuminoid Candy Composition

The following provides an illustrative example for producing a curcuminod candy composition. The method of preparing the candy includes the following steps:
a) Mixing sucrose, liquid glucose and water in a syrup preparation tank
b) Heating the mixture to 100-110° C. to prepare a clear syrup
c) Transferring the syrup of step b to main cooking vessel and heating to 130-150° C.
d) Vaccumising the bulk (800-900 mbar) to achieve water content below 2.5%.
e) Adding an effective concentration of curcumnioids, acidifying and flavouring agents to step d and heating to 125-135° C.
f) Mixing the solution of step e to form a uniform homogenous mixture
g) Subjecting the mixture of step f to cooling & kneading
h) Forwarding the mixture of step g to batch former followed by rope former
i) Passing the rope through candy formation machine to mould the candies
j) Passing the moulded candies through a cooling tunnel at 2-8° C.
l) Packing the solidified chewable candy into suitable containers and packets
   Table 2 provides an illustrative examples of a curcuminoid candy formulation

| | | % | |
|---|---|---|---|
| Sr. No. | Ingredients | Overages | Actual Qty (mg/candy) |
| 1. | Curcumin C3 Complex | 1.05% | 26.25 |
| 2. | Sucrose | 54% | 1350.00 |
| 3. | Liquid Glucose | 44.27% | 1106.75 |
| | | | (Considering 80% solid |
| | | | content.. qty - 1383.44) |
| 4. | Flavour | 0.4% | 10.00 |
| 5. | Menthol | 0.08% | 2.00 |
| 6. | Acidulants (Citric acid/ | 0.2% | 5.00 |
| | Tartaric Acid/Maleic | | |
| | Acid) | | |
| 7. | Purified water | — | q.s. (appx 350) |
| | Total | 100% | 2500.00 |

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method for the manufacture of gummy composition containing curcuminoids, said method comprising steps of:
a) Mixing powdered chewable base in water with constant stirring to obtain a turbid solution;
b) Adding appropriate concentration of sweetners to the solution of step a with continuous stirring,
c) Mixing the solution of step b with suitable acidifying agents and flavouring agents to obtain a colloidal mixture;
d) Heating the mixture of step c to 55° C.-80° C. for 15-20 minutes
e) Adding an effective concentration of curcumnioids to the solution of Step d with continuous stirring to form a uniform mixing of the ingredients
f) Pouring the syrup of step e into moulds of different shapes and sizes that is precoated with corn starch
g) Allowing the syrup to cool at 10-15° C. within the mould and solidify to obtain a chewable gummy
h) Passing the gummies of step g on Vibrosifters to remove excess corn starch
i) Sprinkling suitable sweetners, acidifying agents and flavouring agents as a coating.
j) Packing the solidified chewable candy into suitable containers and packets

2. The method as in claim 1, wherein the chewable base is selected from the group consisting of gelatin, soft gum, nougat and caramel.

3. The method as in claim 1, wherein the chewable base is present at concentrations of 5-80% w/w.

4. The method as in claim 1, wherein the curcuminoids are present at concentrations of 10-250 mg and 1-20% w/w of the total composition.

5. The composition as in claim 4, wherein the ratio of curcuminoids is 75-81% curcumin, 15-19% demethoxy curcumin, and 2.2-6.5% bis demethoxy curcumin.

6. The method as in claim 1, wherein the sweetner is selected from the group consisting of aspartame, sucrose, fructose, glucose, corn syrup, maltose, dextrose, monk fruit extract and sugar substitutes like stevia at concentrations of 30-70% w/w of the total composition.

7. The method as in claim 1, wherein the acidifying agent is selected from the group consisting of citric acid, tartaric acid, fumaric acid and lactic acid, at concentrations of 0.5-10% w/w of the total composition.

8. The method as in claim 1, wherein the flavouring agent is selected from the group consisting of natural flavours obtained from fruits, berries, honey, molasses, maple sugar, or artificial flavours: methyl anthranilate and ethyl caproate, at concentrations of 0.5-10% w/w of the total composition.

9. A chewable gummy composition comprising of effective concentration of curcuminoids, chewable base, acidifying agents, preservatives and flavouring agents.

10. The curcuminoid composition as in claim 9, wherein the composition is more palatable and bioavailable.

11. The curcuminoid composition as in claim 9, wherein the ratio of curcuminoids is 75-81% curcumin, 15-19% demethoxy curcumin, and 2.2-6.5% bis demethoxy curcumin.

12. A method for the manufacture of candy composition containing curcuminoids, said method comprising steps of:
a) Mixing one or more sweetners in water in a syrup preparation tank
b) Heating the mixture to 100-110° C. to prepare a clear syrup
c) Transferring the syrup of step b to main cooking vessel and heating to 130-150° C.
d) Vaccumising the bulk (800-900 mbar) to achieve water content below 2.5%.
e) Adding an effective concentration of curcumnioids, acidifying and flavouring agents to step d and heating to 125-135° C.
f) Mixing the solution of step e to form a uniform homogenous mixture
g) Subjecting the mixture of step f to cooling & kneading
h) Forwarding the mixture of step g to batch former followed by rope former
i) Passing the rope through candy formation machine to mould the candies
j) Passing the moulded candies through a cooling tunnel at 2-8° C.
k) Packing the solidified chewable candy into suitable containers and packets

13. The method as in claim 12, wherein the curcuminoids are present at concentrations of 10-100 mg and 1-20% w/w of the total composition.

14. The method as in claim 12, the ratio of curcuminoids is 75-81% curcumin, 15-19% demethoxy curcumin, and 2.2-6.5% bis demethoxy curcumin.

15. The method as in claim 12, wherein the sweetner is selected from the group consisting of aspartame, sucrose, fructose, glucose, corn syrup, maltose, dextrose, monk fruit extract and sugar substitutes like stevia at concentrations of 30-80% w/w of the total composition.

16. The method as in claim 12, wherein the acidifying agent is selected from the group consisting of citric acid, tartaric acid, fumaric acid and lactic acid, at concentrations of 0.1-10% w/w of the total composition.

17. The method as in claim 12, wherein the flavouring agent is selected from the group consisting of natural flavours obtained from fruits, berries, honey, molasses, maple sugar, or artificial flavours: methyl anthranilate and ethyl caproate, at concentrations of 0.1-10% w/w of the total composition.

18. A chewable candy composition comprising of effective concentration of curcuminoids, chewable base, suitable lubricants, acidifying agents, preservatives and flavouring agents.

19. The curcuminoid composition as in claim 18, wherein composition is more palatable and bioavailable.

20. The curcuminoid composition as in claim 18, the ratio of curcuminoids is 75-81% curcumin, 15-19% demethoxy curcumin, and 2.2-6.5% bis demethoxy curcumin.
